(19) United States
(12) Patent Application Publication
(10) Pub. No.: US 2023/0226079 A1
(43) Pub. Date: Jul. 20, 2023

(54) METHODS AND COMPOSITIONS FOR TREATMENT OF SOLID TUMORS

(71) Applicant: Saint Louis University, St. Louis, MO (US)

(72) Inventors: Ratna Ray, St. Louis, MO (US); Subhayan Sur, St. Louis, MO (US)

(73) Assignee: Saint Louis University, St. Louis, MO (US)

(21) Appl. No.: 18/154,810

(22) Filed: Jan. 14, 2023

Related U.S. Application Data
(60) Provisional application No. 63/299,938, filed on Jan. 15, 2022.

(51) Int. Cl. A61K 31/575; A61K 47/20; A61K 47/40; A61P 35/00

(52) U.S. Cl. CPC A61K 31/575; A61K 47/20; A61K 47/40; A61P 35/00

(57) ABSTRACT

This invention relates to a novel method of treating solid tumors of the head and neck. More specifically, the invention relates to the use of Momordicine-I (M-I), an extract of bitter melon (Momordica charantia), to treat solid tumors, particularly those of the head and neck as well as the breast. Methods and compositions are disclosed related to administrating Momordicine-I with lipophilic solvents to subjects in need.

### RELATED APPLICATIONS

This patent application claims priority from U.S. provisional patent application Ser. No. 63/299,938, filed Jan. 15, 2022, which is incorporated herein by reference in its entirety.

### GOVERNMENT SUPPORT CLAUSE

The work disclosed herein was supported by grant, R01 DE024942 from the National Institute of Health. The U.S. Government has certain rights in this invention.

### FIELD OF THE INVENTION

The invention relates to methods and compositions for treating solid tumors. More specifically, the invention relates to the use of Momordicine-I (M-I), an extract of bitter melon (Momordica charantia), to treat solid tumors in head and neck, or breast, or other solid tumors, for a subject in need.

### BACKGROUND

Head and neck cancer (HNC) arises from the mucosal surfaces of the oral cavity, oropharynx, larynx, paranasal sinuses, nasal cavity, and salivary glands, and is considered the sixth most common cancer worldwide (Vigneswaran et al. (2014) Clin. N. Am.; 26, 123-141). Major risk factors for HNC are habitual tobacco and alcohol consumption, chewing betel quid, chewing tobacco and human papillomavirus (HPV) infection. In 2021, the estimated incidence rate of cancers in the oral cavity and pharynx is 54,010, with an associated 10,850 deaths in the USA (Siegel, et al. (2021) CA Cancer J. Clin.; 71, 7-33). Despite the advancements in surgical techniques, chemotherapy and radiation therapy, treatment of the disease is very challenging for both clinicians and patients. The overall survival rates are 40-50%, which have not improved over the past few decades. Difficulty in performing early diagnosis, lack of early detection markers, adverse side effects, lack of effective chemotherapeutic drugs, therapy resistance and economic expense of conventional therapies make the disease management difficult. The epidermal growth factor receptor (EGFR) inhibitors are the only approved drugs for targeted therapy with limited success and resistance (Rothenberger et al. (2017) Cancers; 9, 39; Alsahafi et al. (2019) Cell Death Dis.; 10, 540). Compensatory activation of another receptor kinase mesenchymal-epithelial transition factor (c-Met) potentially reduces the efficacy of anti-EGFR inhibitors (Rothenberger et al. (2017) Cancers; 9, 39). Aberrant activation of c-Met signaling is frequently observed in HNC and is associated with poor prognosis and metastasis (Rothenberger et al. (2017) Cancers; 9, 39; Alsahafi et al. (2019) Cell Death Dis.; 10, 540; Arnold et al. (2017) Cancers; 9, 169). The c-Met signaling shares common down-stream targets with EGFR and induces HNC cell proliferation, migration, invasion, and metastasis. Several agents that target c-Met have been developed, and results appear promising in HNC preclinical studies. Clinical studies using c-Met inhibitors and monoclonal antibodies are in developing stages. Programmed cell death receptor (PD-1) monoclonal antibodies nivolumab and pembrolizumab were approved in 2016 to treat the advanced and therapy resistance cases (Rothenberger et al. (2017) Cancers; 9, 39; Alsahafi et al. (2019) Cell Death Dis.; 10, 540). However, PD-1 monotherapy generates adaptive resistance and takes a longer time to achieve clinical response than other conventional therapies (Mei et al. (2020) Int. J. Oral Sci.; 12, 16). Thus, the development of additional therapeutic strategies is necessary for successfully managing the disease.

Phytochemicals and their derivatives from plant or microbial sources are promising alternative therapeutic option. While conventional therapy shows limited success due to resistance and adverse side effects, several natural products have showed promising results in preclinical studies (Nerurkar et al. (2010) Pharm. Res.; 27, 1049-1053; Wang et al. (2012) Med. Chem.; 12, 1281-1305; Choudhari et al. (2019) Pharmacol.; 10, 1614). Some phytochemicals have been reported to be able to target multiple molecules in signaling pathways for the control of cancer cell growth and cancer prevention, while being inexpensive and devoid of toxic effects (Wang et al. (2012) Med. Chem.; 12, 1281-1305; Choudhari et al. (2019) Pharmacol.; 10, 1614). Many clinical trials using bioactive secondary metabolites are completed with promising outcomes and some are ongoing. Many drugs derived from plant or microbial sources like vinca alkaloids (vinblastine, vincristine, vindesine, vinorelbine), taxanes (paclitaxel, docetaxel), podophyllotoxin and its derivations (topothecan, irinothecan), anthracyclines (doxorubicin, daunorubicin, epirubicin, idarubicin) are already approved (Choudhari et al. (2019) Pharmacol.; 10, 1614; Safarzadeh et al. (2014) Adv. Pharm. Bull.; 4, 421-427).

The Inventors and others have evaluated the potential anticancer effect of bitter melon (Momordica charantia) extract (BME) in several cancers (Dandawate et al. (2016) Chin. J. Nat. Med.; 14, 81-100; Raina et al. (2016) Semin. Cancer Biol.; 40-41, 116-129; Jia et al. (2017) Int. J. Mol. Sci.; 18, 2555; Muhammad et al. (2017) Oncotarget; 8, 66226-66236; Fang et al. (2019) Curr. Protein Pept. Sci.; 20, 296-301; Sur et al. (2020) Cancers; 12, 2064). In HNC preclinical models, BME prevents HNC cell proliferation targeting c-Met and downstream signaling, inhibits glucose and lipid metabolism, induces cell death, and enhances the immune defense system (Rajamoorthi et al. (2013) PLoS ONE; 8, e78006; Bhattacharya et al. (2016) Oncotarget; 7, 33202-33209; Bhattacharya et al. (2017) Cancer Prey. Res.; 10, 337-344; Sur et al. (2017) Cancer Prey. Res.; 11, 191-202; Sur et al. (2019) Cell Commun. Signal.; 17, 131). Bitter melon belongs to the family Cucurbitaceae, and is cultivated in tropical and sub-tropical regions of Asia, Africa, and South America. The plant has the highest nutritional values among other cucurbits. It contains diverse secondary metabolite classes, including cucurbitane type triterpenes, phenolic acids, flavonoids, essential oils, sterols, saponin, and primary metabolites, including fatty acids, amino acids, lectins, and some proteins (Dandawate et al. (2016) Chin. J. Nat. Med.; 14, 81-100; Raina et al. (2016) Sem in. Cancer Biol.; 40-41, 116-129; Sur et al. (2020) Cancers; 12, 2064). Among the isolated compounds, momordica antiviral protein, 30 kD (MAP30), momorcharin, RNase MC2, kuguacin J, eleostearic acids and lectins showed anticancer effect in different models (Sur et al. (2020) Cancers; 12, 2064). However, limited follow-up studies were reported with the compounds in preclinical models. This disclosure will identify the bioactive metabolite(s) from the BME and evaluate its role in HNC preclinical mouse model. The Inventors' results highlighted momordicine-I (M-I) as a potent active component in the BME. M-I is non-toxic, stable in blood and acts similarly to the extract as HNC growth inhibitor involving c-Met and downstream signaling in both in vitro and in vivo models. This is the first study describing the therapeutic potential of M-I for the regression of HNC tumors in a pre-clinical model. The Inventors has also demonstrated these methods are useful for treating solid tumors in a breast cancer model.

### SUMMARY OF THE INVENTION

A method of treating a subject with a solid tumor, comprising, selecting a subject diagnosed with a solid tumor and administering a pharmaceutical solution comprising one or more lipophilic solvents and an effective amount of Momordicine-I.

A method of treating a subject with head and neck cancer, comprising, selecting a subject diagnosed with head and neck cancer and administering a pharmaceutical solution comprising one or more lipophilic solvents and an effective amount of Momordicine-I.

A method of treating a subject with breast cancer by administering a pharmaceutical solution comprising one or more lipophilic solvents and an effective amount of Momordicine-I, whereas symptoms or indicators by way of example, pSTAT3, and/or ki67 are diminished.

A composition for the treatment of head and neck cancer, the composition comprising, a pharmaceutical acceptable composition of one or more lipophilic solvents and an effective amount of Momordicine-I.

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

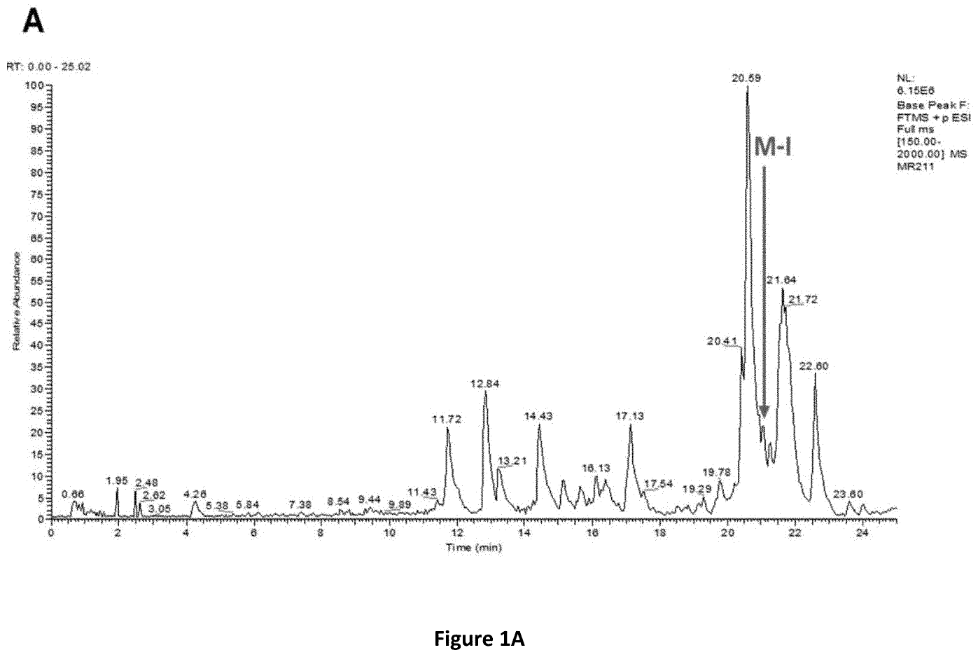

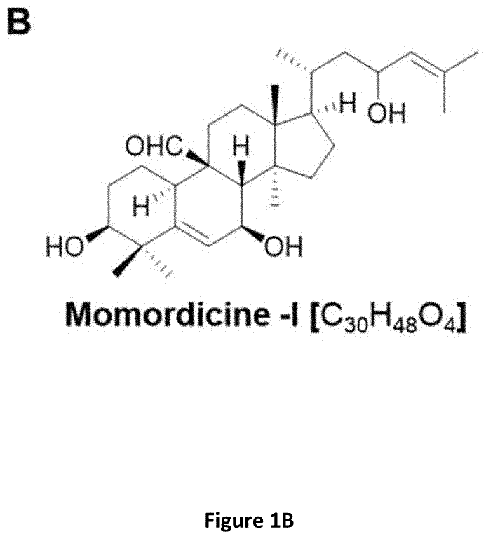

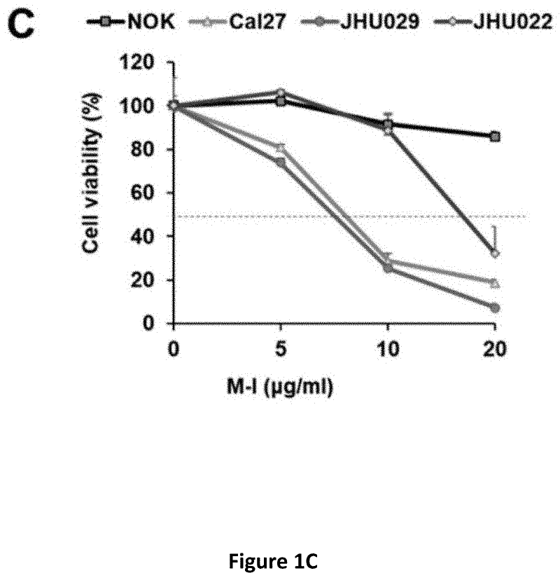

FIGS. 1A-1C illustrates Mass-spectrometric analysis of bitter melon extract and identification of momordicine-I. FIG. 1A illustrates a LC trace of bitter melon extract done by HRMS. The red arrow indicated the presence of momordicine-I (M-I) peak. FIG. 1B illustrates the chemical structure of momordicine-I.

FIG. 1C illustrates HNC (Ca127, JHU029, JHU022) and control NOK cells treated with M-I at different concentrations for 48 h, and cytotoxicity assay performed. Small bar indicates standard error.

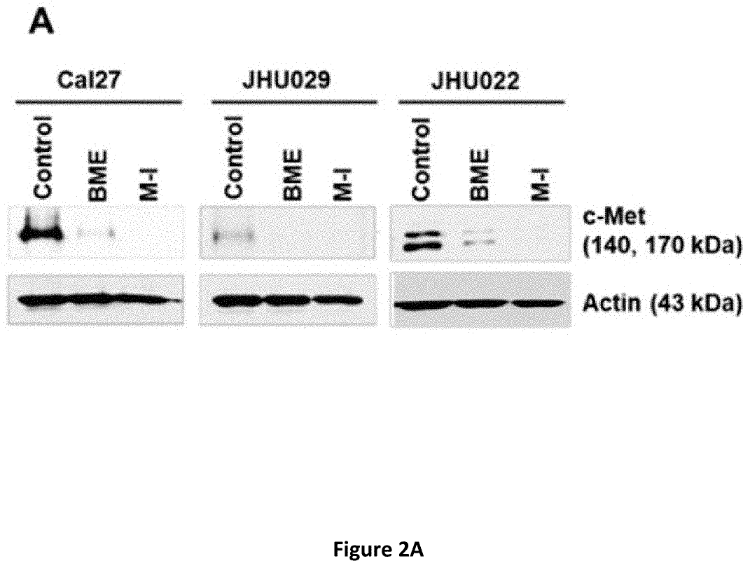

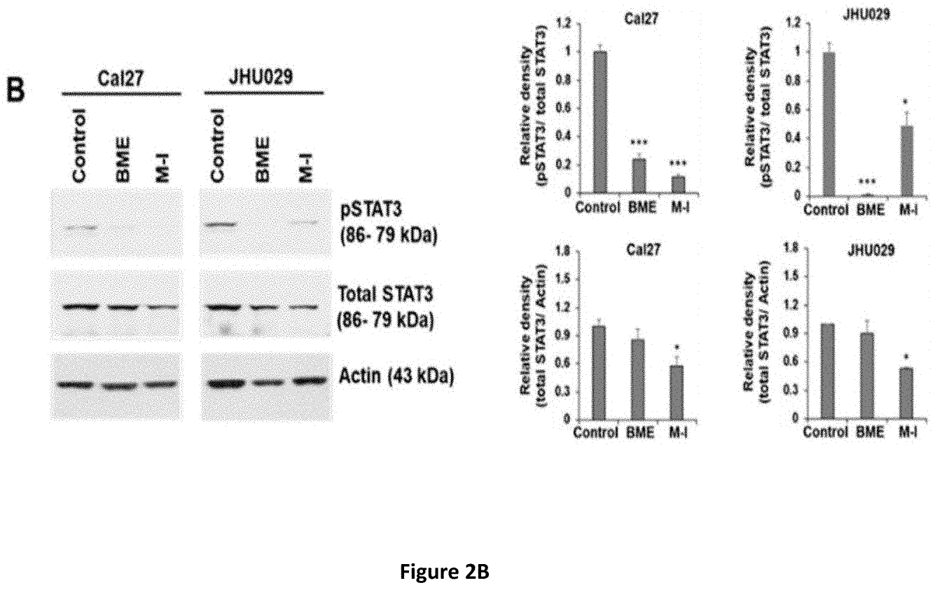

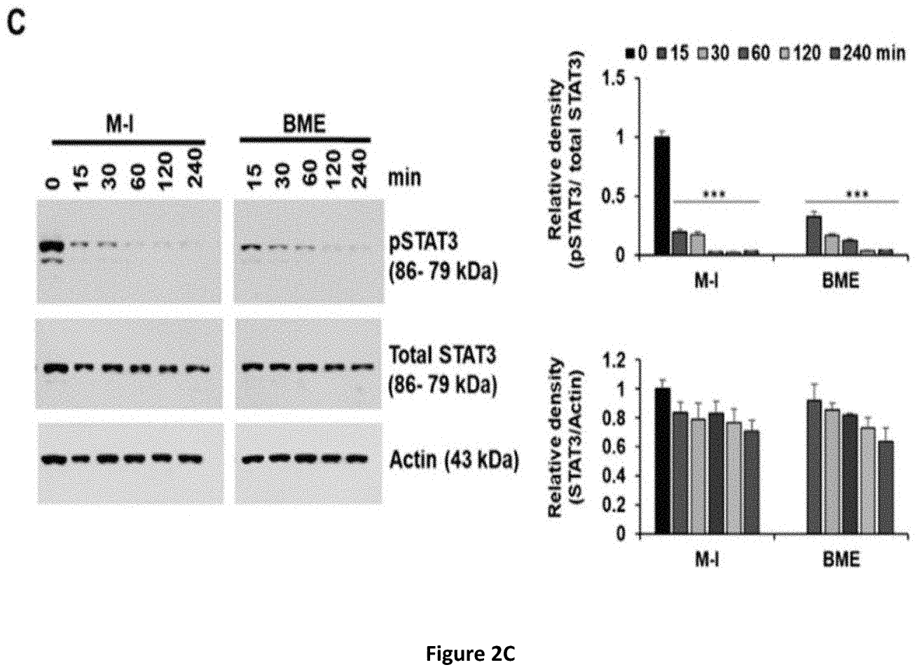

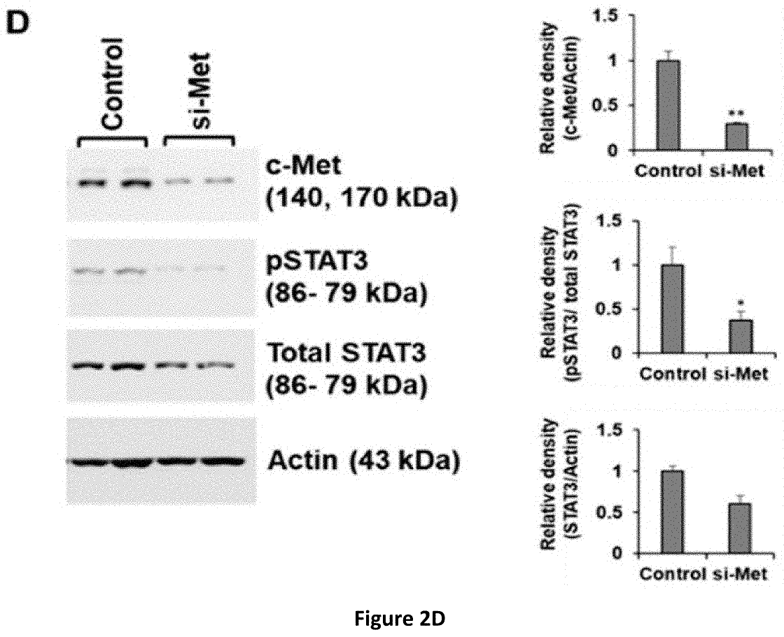

FIGS. 2A-2D illustrates Momordicine-I inhibited c-Met signaling in HNC cells. FIG. 2A illustrates Ca127, JHU029 and JHU022 cells were treated with either 2% BME or 10 ug/mL of M-I and JHU022 cells were treated with either 3% BME or 20 ug/mL dose of M-I. Cell lysates were prepared after 48 hr of treatment and subjected to Western blot analysis using specific antibodies. Representative Western blot images for c-Met expression in Ca127, JHU029 and JHU022 cells are shown. Membrane was reprobed with the antibody for actin as an internal control. FIG. 2B illustrates a representative Western blot image for phospho-STAT3 (pSTAT3 Tyr-705) and total STAT3 expression in Ca127 and JHU029 cells with BME or M-I treatment. Membrane was reprobed by actin as an internal control. Quantitative representation of Western blot band intensities (right panel). Small bar indicates standard error (*p<0.05; *** p<0.001). FIG. 2C illustrates Ca127 cells treated with 2% BME or M-I (10 ug/mL) at indicated time points. FIG. 2D illustrates Ca127 cells treated with control siRNA or siRNA to c-Met (si-Met) for 48 hr. Cell lysates (from panels C and D) were subjected to analyze by western blot for pSTAT3 or STAT3 using specific antibody. Membranes were reprobed by actin as internal control. Quantitative representation of Western blot band intensities (right panel). Small bar indicates standard error (*** p <0.001).

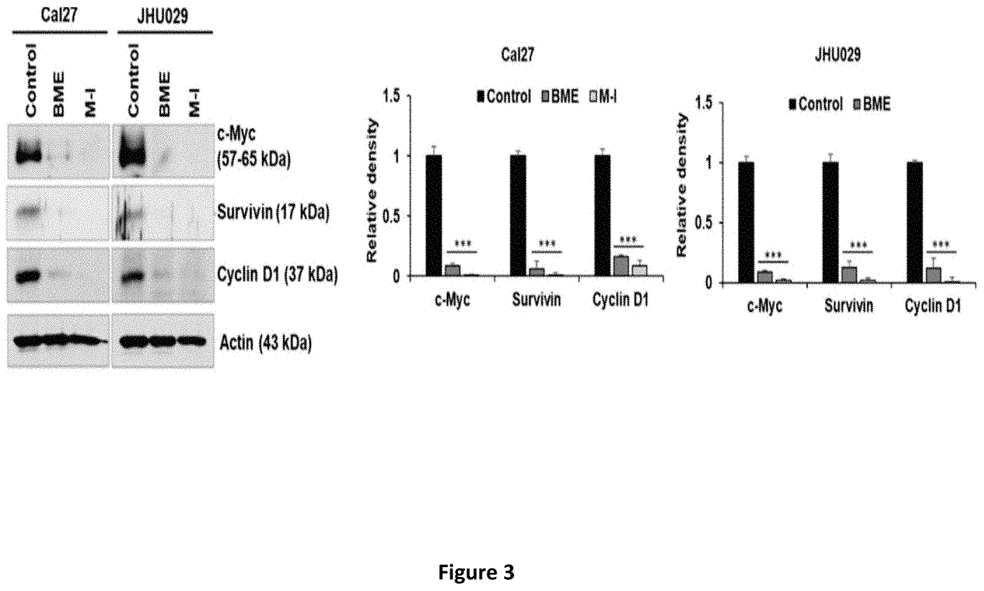

FIG. 3 illustrates how Momordicine-I inhibits downstream of c-Met signaling in HNC cells. Representative Western blot image for c-Myc, survivin and cyclin D1 expression in Ca127 and JHU029 cells with treatment of BME or M-I. The same membrane used in panel A was reprobed. Actin was used as internal control. Quantitative representation of Western blot band intensities (right panel). Small bar indicates standard error (***p<0.001).

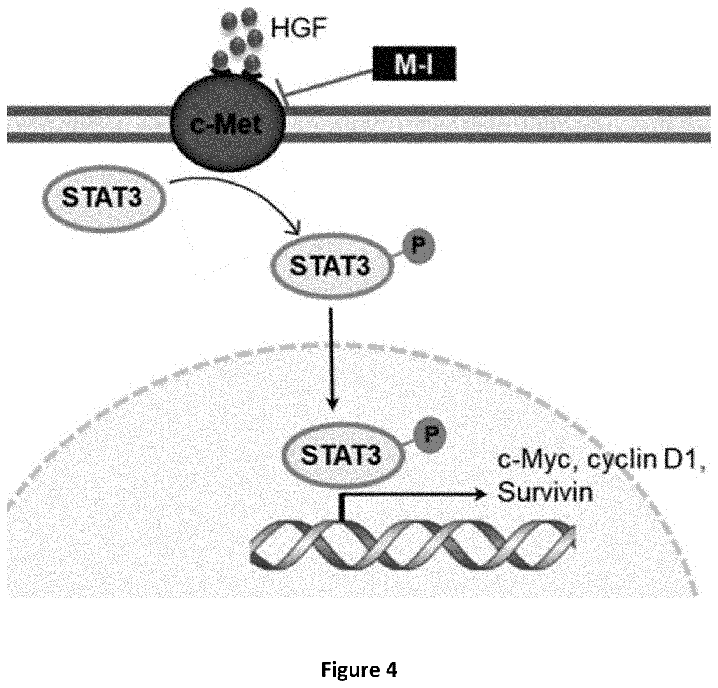

FIG. 4 illustrates a schematic representation showing mode of action of M-I in inhibition of c-Met signaling. Sharp arrows indicate activation/ induction and blunt arrows indicate inhibition.

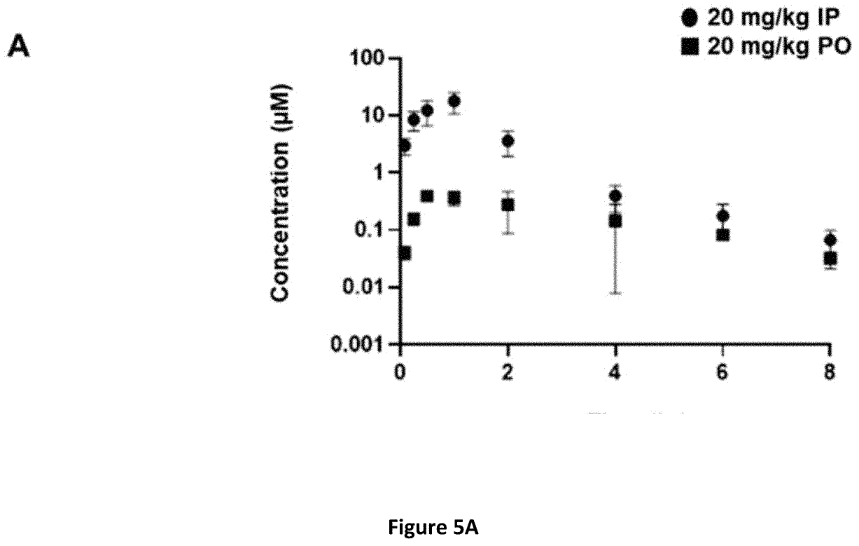

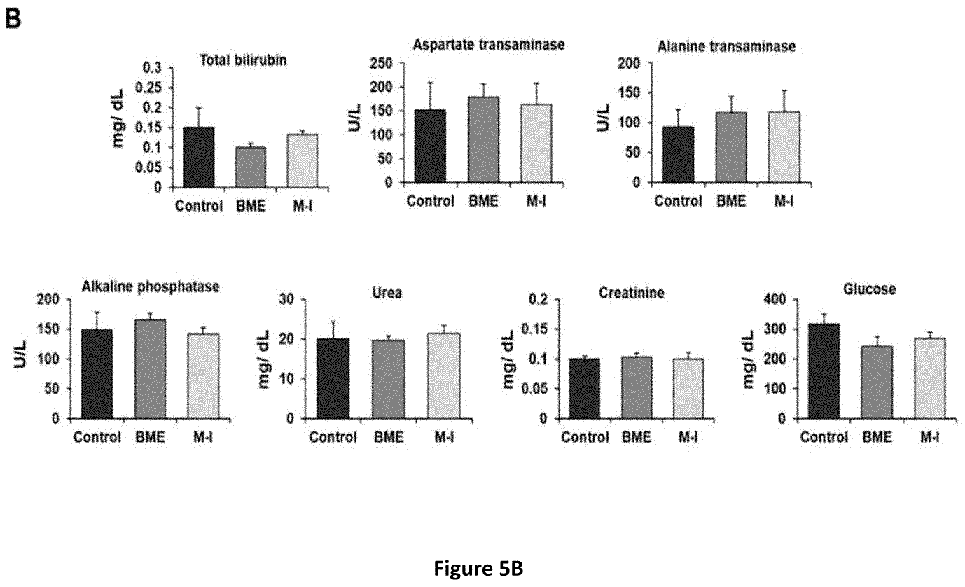

FIGS. 5A-5B illustrates the in vivo pharmacokinetic and toxicity profile of momordicine-I. FIG. 5A illustrates concentration-time profile of momordicine-I in C57131/6 male mice at 20 mg/kg single dose administered by IP injection or oral gavage (PO). Data is from n=3 mice and represents mean standard deviation. FIG. 5B illustrates a comparison of metabolic panels from serum among M-1(30 mg/kg/mouse) and BME treated groups with untreated control mice. Small bar indicates standard error.

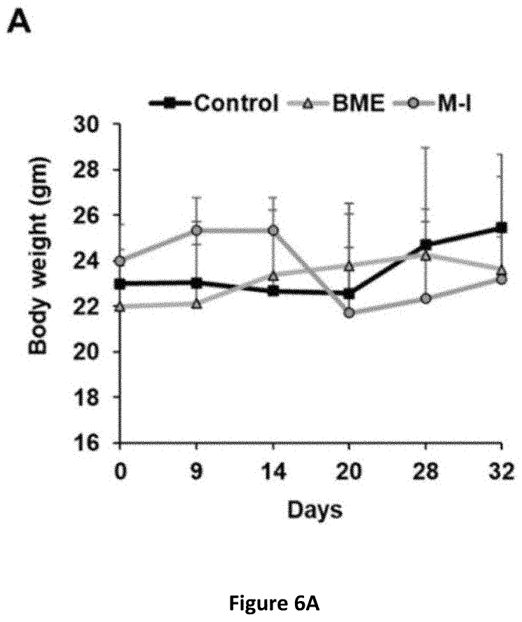

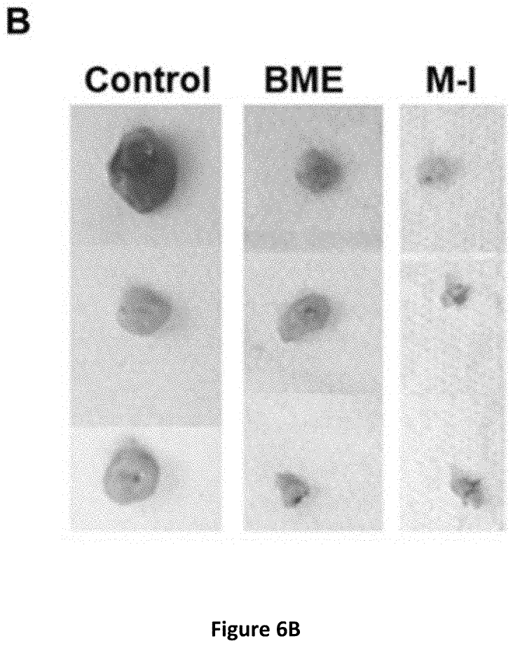

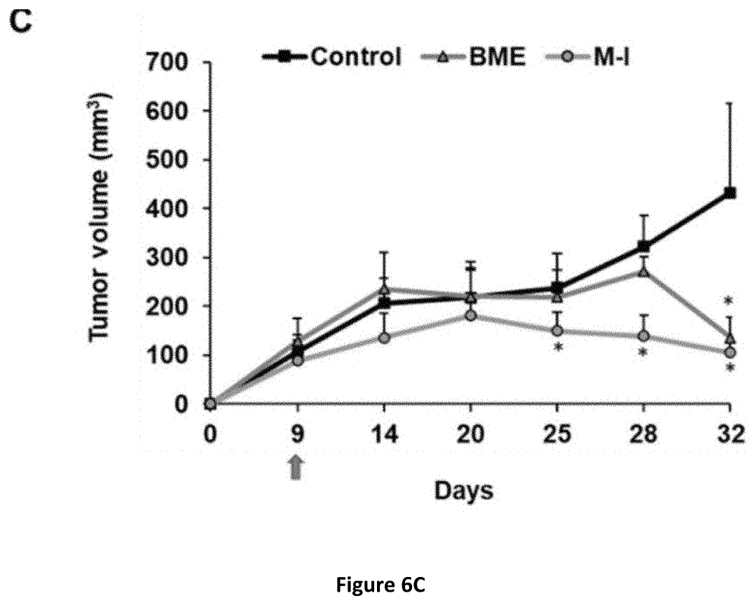

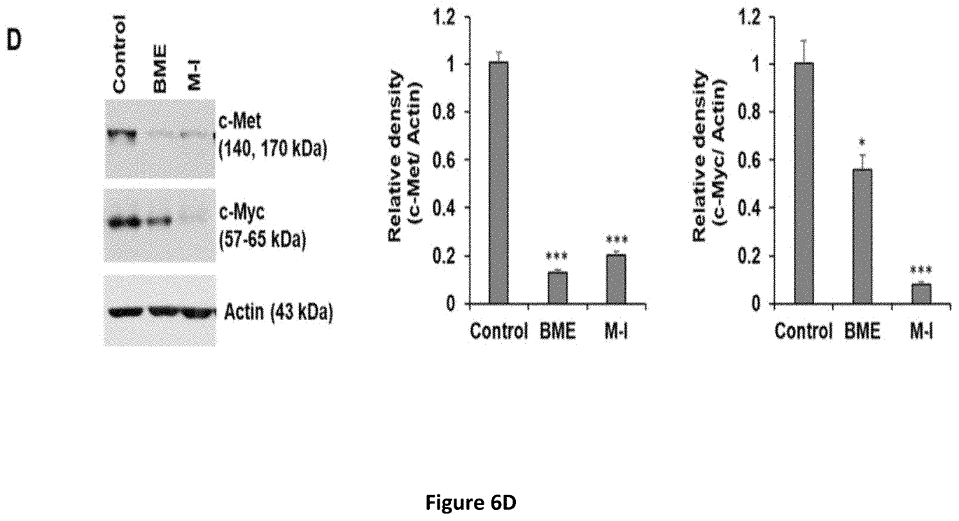

FIGS. 6A-6D illustrates the therapeutic effect of momordicine-I in JHU029 xenograft model. JHU029 cells (1.5×106) cells were injected subcutaneously into the flank of nude mice. After the formation of a palpable tumor, mice were randomly divided into three groups (n=5): Control (without any treatment), BME group (30% BME through drinking water) and M-I group (30 mg/kg IP, once in a day and every day). FIG. 6A illustrates body weight measured in control and treated mice. FIG. 6B illustrates representative images of tumors in control and treatment groups. FIG. 6C illustrates tumors measured using a slide caliper and tumor volumes calculated. Arrow indicates starting point BME/M-I treatment. FIG. 6D illustrates control or treated tumor lysates subjected to Western blot analysis for c-Met and c-Myc expression using specific antibodies and representative bands shown. The blot was reprobed with an antibody to Actin for normalization. Right panel shows quantitation. Small bar indicates standard error (*, p<0.05; **, p<0.01; *** p<0.001).

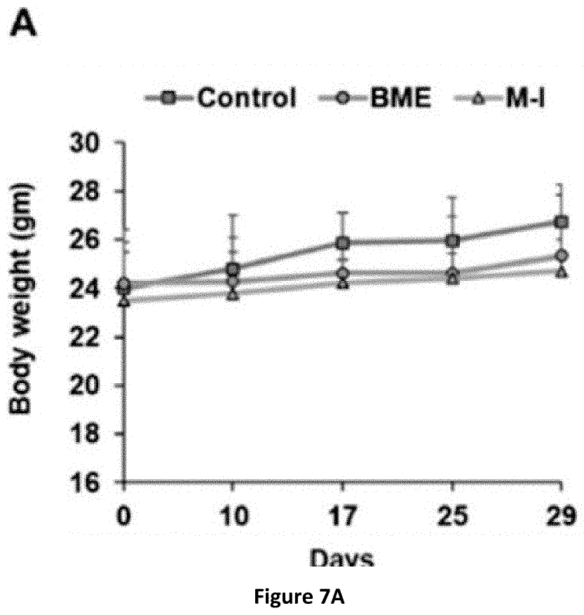

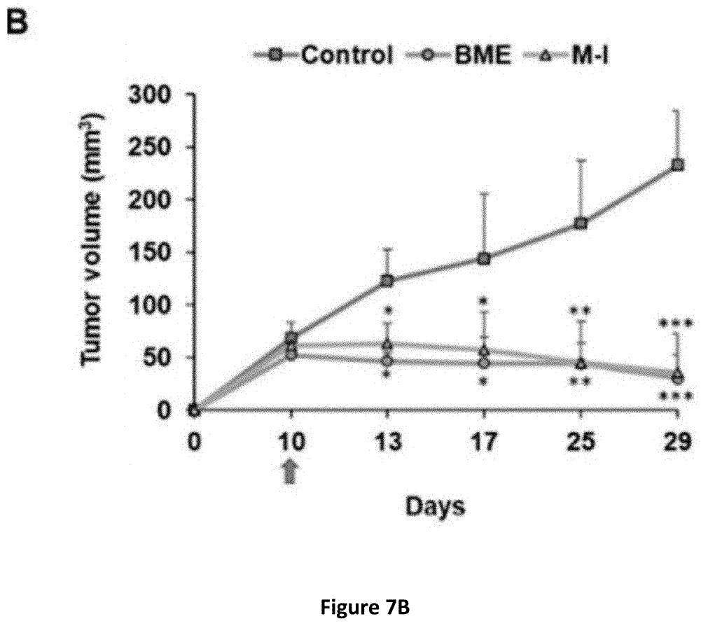

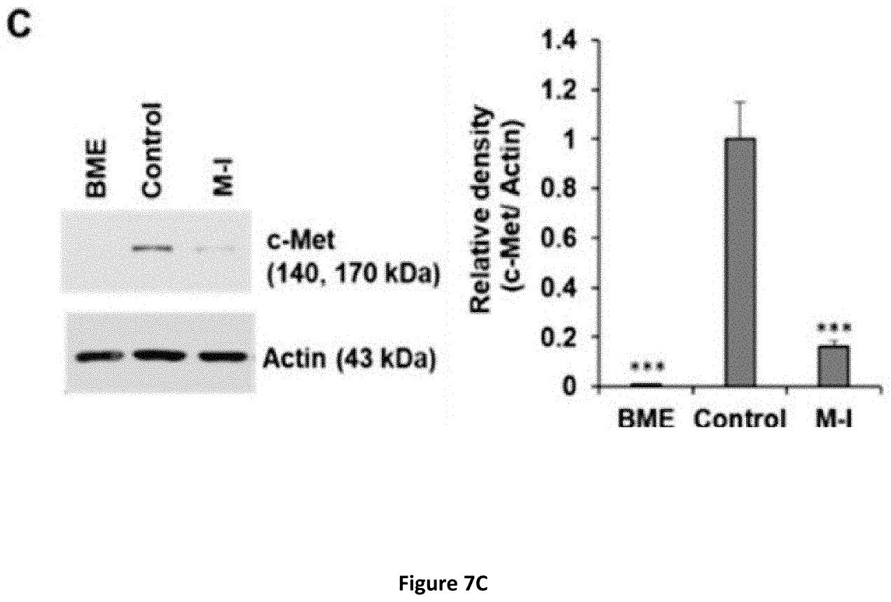

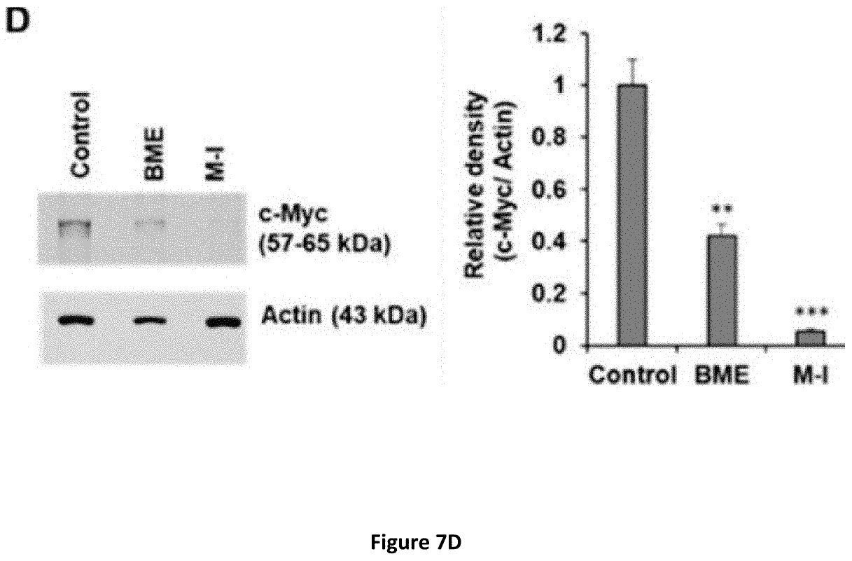

FIGS. 7A-7D illustrates the therapeutic effect of momordicine-I in Ca127 xenograft model. Ca127 cells (1.5×106) cells were injected subcutaneously into the flank of nude mice. After formation of palpable tumor, mice. Therapeutic effect of momordicine-I in Ca127 xenograft model. Ca127 cells (1.5 106) cells were injected subcutaneously into the flank of nude mice. After formation of palpable tumor, mice were randomly divided into three groups (n=5): Control (without any treatment), BME group (30% BME through drinking water) and M-I group (30 mg/kg IP, once in a day and every day). FIG. 7A illustrates body weight measured in control and treated mice. FIG. 7B illustrates tumors measured using a slide caliper and tumor volumes were calculated. Arrow indicates the starting point BME/ M-I treatment. FIGS. 7C and FIG. 7D illustrate Control and treated tumor lysates respectively subjected to Western blot analysis for c-Met and c-Myc expression using specific antibodies and representative bands are shown. The blot was reprobed with an antibody to Actin for normalization. Right panel shows quantitation. Small bar indicates standard error (*, p<0.05; **, p<0.01; *** p<0.001).

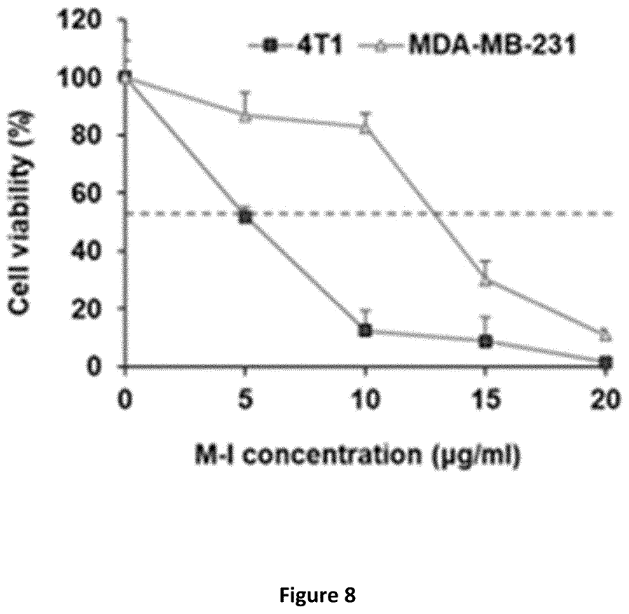

FIG. 8 illustrates 4T1 and MDA-MB-231 cells treated with M-I at indicated doses for 72h. The viability of control cells was arbitrarily set to 100%. Results shown are an average of three independent experiments. Data are represented as mean±SD.

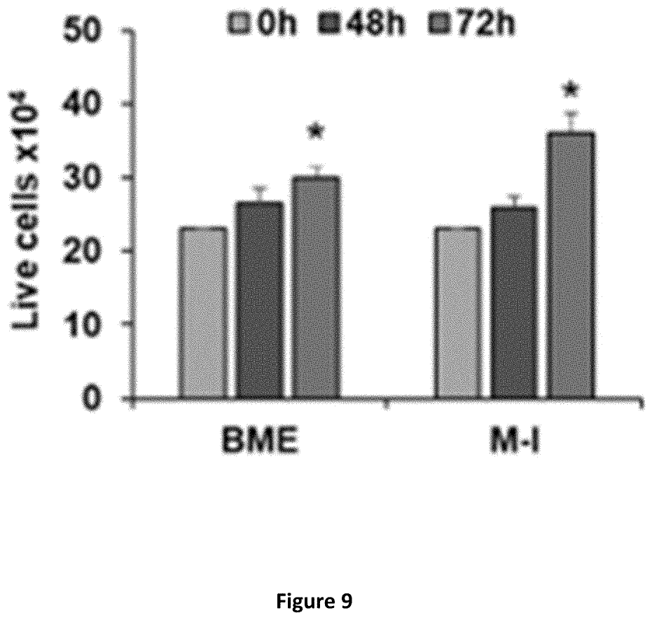

FIG. 9 illustrates NK3.3 cells treated with bitter melon extract or M-I and cell viability was measured at 48h or 72h after treatment. Results shown are an average of three independent experiments. Small bar indicates standard error. (* p <0.05)

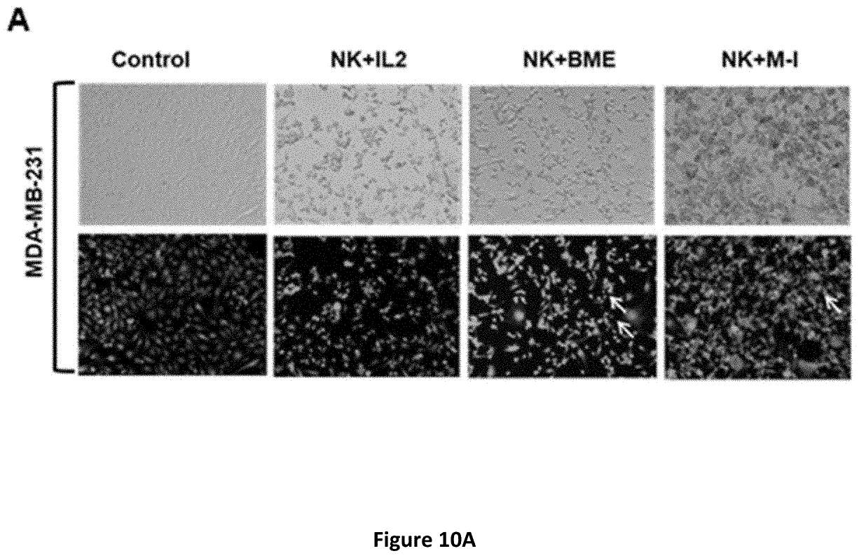

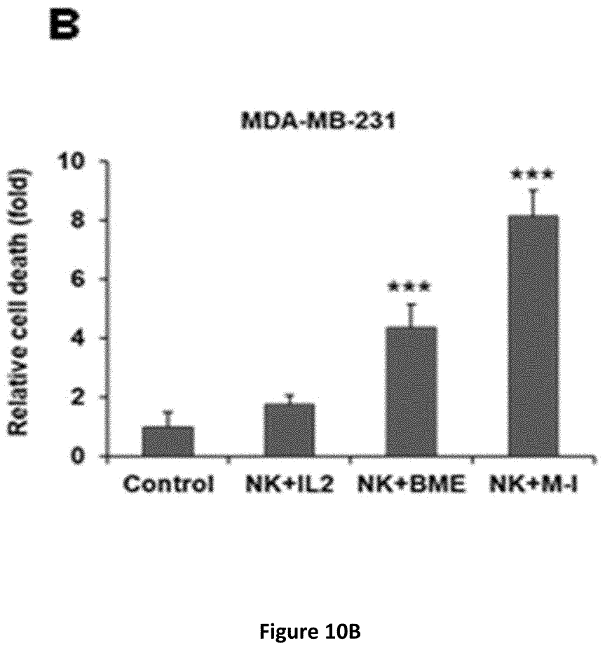

FIG. 10A illustrates Treated or untreated cells were stained with Calcein AM (green color indicates live cells) and ethidium homodimer-1 (red color inndicates dead cells-shown by white arrows) to quantitate the live and dead cells by fluorescence microscopy with corresponding bright field images. Magnification 10X. Arrows indicate dead cells. FIG. 10B illustrates Quantitation of relative dead cells, calculated from five random fields. Small bar indicates standard error ***p<0.001).

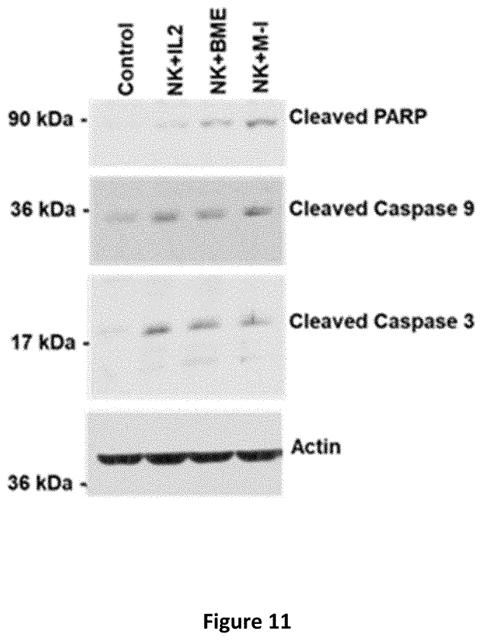

FIG. 11 illustrates M-I activates NK cells for killing activity. MDA-MB-231 cells were co-cultured with NK cells activated by IL2, BME or M-I. After 24 hr cell lysates were prepared and subjected to Western blot analysis using specific antibodies. Left panel: Representative western blot images for cleaved PARP, expression of cleaved Caspase 9 and 3 are shown. Membrane was reprobed with antibody for Actin as an internal control.

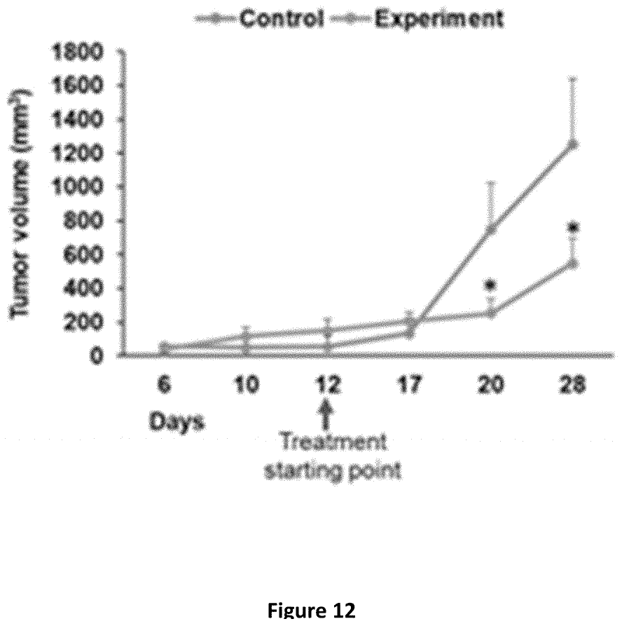

FIG. 12 illustrates 4T1 cells tumor bearing mice were randomly divided into two groups mice (n=4). Starting day of treatment by M-I (red color) or vehicle (control-blue color) graph was marked by up arrow.

### DETAILED DESCRIPTION OF THE INVENTION

The Inventors have identified Momordicine-I (M-I) as a bioactive secondary metabolite using LC-HRESIMS analysis of the BME. They have demonstrated that M-I inhibits c-Met signaling in HNC cell (JHU022, JHU029, Ca127) lines, as well as a significant regression of tumor growth in both HNC and breast cancer xenograft models following daily administration of M-I with no toxicity. While not wishing to be bound by theory, the Inventors believe that the therapeutic efficacy of BME and M-I is facilitated by targeting c-Met signaling.

Bitter melon plant and its fruit contain many phytochemicals, and among those cucurbitane type triterpenoids and cucurbitane type triterpene glycosides are the major chemical constituents (Sur et al. (2020) Cancers; 12, 2064). The cucurbitane-type triterpenoids and cucurbitane-type triterpene glycosides are suggested to be responsible for bitterness and confer much of the biological activities of the plant (Sur et al. (2020) Cancers; 12, 2064; Wu et al. (2014) Planta Medica; 80, 907-911). M-I belongs to the class cucurbitane-type triterpene. This secondary metabolite was first identified and characterized in leaves and vines of Momordica charantia L. (Yasuda et al. (1984) L. Chem. Pharm. Bull.; 32, 2044-2047). The Inventors identified M-I in the water extract of the fruit. Although the compound was identified in 1984, the biological function of M-I was not well evaluated. M-I was recently reported to have inhibitory effects on high-glucose-induced cell proliferation and collagen synthesis in rat cardiac fibroblasts (Chen et al. (2018) Oxidative Med. Cell. Longev; 3939714) and stimulate insulin secretion in vitro (Keller et al. (2011) Phytomedicine; 19, 32-37), but to the best of the Inventors' knowledge, the anticancer effect of M-I has not been reported.

The Inventors initially screened a few metabolites from bitter melon and observed a significant inhibition with M-I with IC50 of less than 8 ug/mL in Ca127 and JHU029 cells. The Inventors also observed that M-I inhibited HNC growth and c-Met signaling. Aberrant activation of c-Met signaling through overexpression of c-Met and its downstream molecules c-Myc, cyclin D1, and survivin were observed predominantly in HNC (Rothenberger et al. (2017) Cancers; 9, 39; Alsahafi et al. (2019) Cell Death Dis.; 10, 540; Arnold et al. (2017) Cancers; 9, 169), which were reduced following M-I treatment. Increased c-Met signaling is associated with HNC progression and metastasis, and c-Met signaling inhibition by neutralizing antibody inhibited tumor growth and its metastatic potential (Rothenberger et al. (2017) Cancers; 9, 39; Arnold et al. (2017) Cancers; 9, 169). M-I may have other targets to inhibit cell proliferation which was not investigated in this study. The Inventors observed a significant reduction of tumor growth in two HNC xenograft models following BME treatment with no toxicity. Furthermore, the Inventors demonstrated that treatment of M-I (30 mg/kg/mouse) once a day worked similarly to twice a day in the Inventors' pilot experiment (20 mg/kg/mouse).

Head and neck cancer (HNC) therapy often has limited success. Resistance to approved anti-EGFR therapy sometimes makes the treatment management difficult (Rothenberger et al. (2017) Cancers; 9, 39; Arnold et al. (2017) Cancers; 9, 169). Thus, targeting c-Met is suggested to be a promising alternative strategy. Many drugs that target c-Met signaling have been developed, showing promising results in preclinical and clinical studies. However, these drugs manifested several adverse side effects (Rothenberger et al. (2017) Cancers; 9, 39; Arnold et al. (2017) Cancers; 9, 169; Hu et al. (2017) Cancers; 9, 58). c-Met inhibitor GEN-203 and compound 8 showed liver and bone marrow toxicity in mice and myocardial degeneration in rats. Foretinib caused fatigue, hypertension, and gastrointestinal toxicities. Golvatinib caused supraventricular tachycardia, convulsion, and pulmonary embolism. Tivantinib (ARQ197) showed adverse events, including leukopenia, anemia, and neutropenia in clinical trial. Thus, identifying a new natural and non-toxic c-Met signaling inhibitor would have a high impact on HNC treatment, and M-I might be one of the major contributors with biological activity in BME.

### I. Momordicine-I (M-I)

Momordicine-I (M-I) also known as Momordicin I (PubChem CID: 101293615) may be isolated from bitter melon (Momordica charantia) or may be obtained commercially. Chemfaces (Cat. No.: CFN92076; Hubei, China) is one example of a commercial source. Isolated M-I may be dissolved in DMSO, ethyl acetate, acetone, methanol, or dichloromethane to create or incorporate into an injectable solution.

### II. Subjects

It is envisioned that subjects selected for treatment would include human subjects with solid tumors, particularly human subjects diagnosed with head and neck cancer (HNC), or breast cancer. It is also expected that subjects would include experimental and domestic animals in need. The M-I of the invention may be administered to subjects with any stage of tumor progression, whether HNC, breast cancer, or other solid tumor. It is expected that the incident of, or symptoms associated with tumor progression, whether HNC, breast cancer, or other solid tumor, will be arrested, reduced, or diminished. The effectiveness of M-I treatment may be assessed by a reduction of symptoms and/or biomarkers or indicators associated with that particular tumor or cancer. In one non-limiting example, HNC may be assessed by biopsy analyzed using the appropriate immune-histochemistry, before and after treatment with M-I. Examples of target antigens which may be employed for immune-histochemistry include C-Met, and/or pSTAT3, and/or ki67, or other markers for cellular proliferation.

### III. Treatment

It is expected that treatment with Momordicine-I (M-I) of invention will arrest, reduce, diminish, or reverse the symptoms and/or spread or further incidence of HNC, breast cancer or other solid tumors. M-I therapy may include any method capable of delivering M-I to the target sites including systemic or targeted administration. By way of non-limiting example, M-I may be administered to subjects, including humans, intravenously, interperitoneally, subdermally, intramuscularly, or orally.

A lipophilic solvent including but not limited to one or more of DMSO, ethyl acetate, acetone, methanol, or dichloromethane, may be used to solubilize purified M-I. The solubilized M-I and lipophilic solvent may then be included in a pharmaceutical acceptable composition for injection or consumption. A stabilizing compound such as cyclodextrin by way of example Captisol may also be included.

A pharmaceutical acceptable composition for injection or consumption is an aqueous solution and may also include a preservative, a composition to block non-specific binding of M-I, by way of example a protein such as gelatin or albumin, or a compound, such as a surfactant, by way of example, Tween 20 preferably at 0.1 to 1 percent. The solution may also include compounds to prevent degradation or aggregation of M-1 while staying within physiological acceptable parameters for injection. By way example, the pH may vary from 5.0 to 6.0, 6.0 to 7.0, 7.0, to 8.0, or 9.0 to 10.0. One or more salts may also be included. By way of non-limiting example, a phycological acceptable concentration of salt may be a concentration, plus or minus up to 5, 10, or 15 percent that normally found in the subject. Non-naturally occurring salts may also be included, by way of example, Tris, HCL. Formulations of Mom ordicine-I may be concentrated to some degree or lyophilized for storage and later hydrated before use.

The aqueous solution may further contain various salts or buffers that are well known in the art. Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are, Ringer's solution, or isotonic sodium chloride solution.

An effective dose of M-I is expected to vary between subjects. The dose per subject is best determined by a skilled practitioner. The skilled practitioner is typically a threating physician. Examples of effective amounts may be: from 0.1 mg/kg to 1 mg/kg, from 1 mg/kg to 5 mg/kg, from 5 mg/kg to 10 mg/kg, from 10 mg/kg to 20 mg/kg, from 20 mg/kg to 30 mg/kg, from 30 mg/kg to 40 mg/kg, from 40 mg/kg to 50 mg/kg, from 50 mg/kg to 60 mg/kg, from 60 mg/kg to 70 mg/kg, from 70 mg/kg to 80 mg/kg, from 80 mg/kg to 90 mg/kg, from 90 mg/kg to 100 mg/kg, and from 100 mg/kg to 1000 mg/kg. A preferable effective amount may be about 20 mg/kg and most preferable effective amount may be about 30 mg/kg.

By way of example, M-I may be administered to subjects, including humans by bolus interperitoneally injections. In one non-limiting example, M-I treatment may be a human subject receiving a 30 mg/kg dose of M-I by bolus interperitoneally injections, once in a day, every day until HNC symptoms or indications are diminished or are no longer detectable. An non-limiting example of an injectable treatment solution used to administer M-I is a solution formulated at 2 mg M-I /mL dissolved in 5% DMSO/95% of a 30% w:v Captisol solution.

In another non-limiting example, a daily 30 mg/kg dose of M-I treatment may be administered to a human by intravenous injection using a treatment solution of 2 mg M-I /mL dissolved in 5% DMSO/95% of a 30% w:v Captisol solution.

Alternatively, or concurrently, BME or M-I or any of the treatment solutions, as described herein may be administered orally. Bitter melons have been shown to be safe and well tolerated in humans as it has been considered an eatable fruit for many years.

Disclosed herein are exemplary methods of treatment. It is expected that effective amounts and specific protocols will vary with different subjects. An effective amount or treatment protocol may be prescribed by the treating physician. Any of the disclosed M-1 treatments may be used alone, together or in combination with other treatments.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Materials and Methods

### Cell Culture

Normal oral keratinocytes (NOK) (kindly gifted by Dr. Karl Mugner, Tuffs University, Boston, MA, USA) were maintained in Keratinocyte SFM medium supplemented with EGF and bovine pituitary extract (GIBCO, Life Technologies, Berkeley, MO, USA) and 1% penicillin/ streptomycin. HNC cell line Ca127 was purchased from the ATCC. JHU029 (JHU-29) and JHU022 (JHU-22) cell lines were procured from the Johns Hopkins University (Baltimore, MD, USA). The Ca127 and JHU022 cells were maintained in Dulbecco's Modified Eagle Medium (DMEM), and JHU029 cells were in RPMI1640 (Sigma, St. Louis, Mo., USA) media supplemented with 10% FBS and 1% penicillin/ streptomycin (Sigma-Aldrich, St. Louis, Mo., USA) in a humidified CO2 incubator. The cell lines are routinely tested in the Inventors' laboratory to rule out mycoplasma contamination using a commercial Mycoplasma Detection kit (MycoAlert™, Lonza, Morrisville, N.C., USA). Ca127 cells are tongue origin and JHU022 and JHU029 cells are from larynx origin.

### Preparation of Bitter Melon Extract (BME) and Momordicine I (M-I)

Bitter melon extract (BME) was prepared from the Chinese variety of young bitter melons (raw and green) as described previously (Rajamoorthi et al. (2013) PLoS ONE; 8, e78006). Briefly, BME was prepared by aqueous extraction from whole fruit without seeds using a household juicer at room temperature with subsequent centrifugation at 15000 g at 4 C for 30 min. BME was stored at −80 C for further analysis. Ca127 and JHU029 cells were treated with 2% BME and JHU022 cells were treated with 3% BME as described previously (Rajamoorthi et al. (2013) PLoS ONE; 8, e78006; Sur et al. (2019) Cell Commun. Signal.; 17, 131). The momordicine-I (>98% pure) was purchased from Chemfaces (Cat. No.: CFN92076; Hubei, China). The powder was dissolved in DMSO and added to the cells at different concentrations. Based on cytotoxicity data, Ca127 and JHU029 cells were treated with 10 ug/mL and JHU022 cells were treated with 20 ug/mL dose of M-I and incubated for 48 hr for further experiments. All the experiments were done at least in triplicate.

### Cytotoxicity Assay

Ca127, JHU029, JHU022 and NOK were seeded in 96 well-plate (5000 cells/well) and cells were treated with different concentration of M-I for 48 h. There were untreated control and DMSO treated vehicle control group for comparison. Cytotoxicity assay was performed using Cell Counting Kit-8 (Dojindo Molecular Technology, Rockville, Md., USA) according to manufacturer instruction.

Protein Isolation and Western Blot Analysis

Lysates from control, BME or M-I treated cells and tumors were prepared using 2 SDS sample buffer and subjected to western blot analysis using specific antibodies to c-Met (1:500, Cell Signaling Technology, CST, Denvers, Mass., USA,), phospho-STAT3 (pSTAT3-Tyr-705) (1:1000, CST), total STAT3 (1:1000, CST), c-Myc (1:1000, CST), surviving (1:500, Santa Cruz Biotechnology, SBT, Dallas, Tex., USA) and cyclin D1 (1: 500, SBT). HRP conjugated anti-mouse or anti-rabbit secondary antibodies were purchased from Bio-Rad (Hercules, Calif., USA). The blot was reprobed with HRP conjugated -actin antibody (1:5000; SBT) to compare protein load in each lane. Densitometry analysis was done using Image J software (NIH, Bethesda, Md., USA).

### Liquid Chromatography High Resolution Electrospray Ionization Mass Spectrometry (LC-HRESIMS)

HRESIMS analysis of the BME was done using a LTQ Orbitrap spectrometer coupled to an HPLC system (PDA detector, PDA autosampler, and pump, ThermoFisher Scientific, Inchinnan, Renfrew PA4 9R, UK). The following conditions were used: capillary voltage of 45 V, capillary temperature of 260 C, auxiliary gas flow rate of 10-20 arbitrary units, sheath gas flow rate of 40-50 arbitrary units, spray voltage of 4.5 kV, and mass range of 100-2000 amu (maximal resolution of 30,000). For LC-HRESIMS, a Sunfire C18 analytical HPLC column (5 m, 4.6 mm 150 mm) was used with a mobile phase of 0 to 100% Me0H over 20 min followed by 100% Me0H over 5 min at a flow rate of 1 mL min⁻¹.

### Pharmacokinetic and Toxicity Study

The pharmacokinetic (PK) study of M-I was performed in male C57B1/6 mice. A single dose of M-I (formulated at 2 mg/mL dissolved in 5% DMSO/95% of a 30% w:v Captisol solution) was administered either by intraperitoneal injection (IP group, n=3) or oral gavage (PO group, n=3). After dosing, 20 uL blood samples were collected into heparin pre-coated tubes at 5 min, 15 min, 30 min, 1 h, 2 h, 4 h, 6 h, 8 h and 24 h. Samples were centrifuged, and the plasma collected. M-I concentration was determined using liquid chromatography-mass spectrometry/mass spectrometry (LC-MS/MS) as described earlier (Fuerst et al. (2018) Chem.; 26, 4984-4995).

For toxicity study, C57B1/6 male mice were received either 30% BME through drinking water or 20 mg/kg of M-I twice a day or 30 mg/kg of M-I once a day by IP injection for 5 days. There were three mice in each group, including untreated control. Blood was collected on day 6, and serum was prepared. Serum parameters related to hepatotoxicity (total bilirubin, alanine transaminase, aspartate transaminase, and alkaline phosphatase) and nephrotoxicity (urea and creatinine) were performed.

### Tumorigenicity Assay

JHU029 or Ca127 cells (1.5 10⁶) containing 40% Matrigel were injected subcutaneously into the flank of BALB/c athymic nude mice (7-8 weeks old). When the palpable tumor was developed (>60 mm3), mice were divided into three groups randomly, 5 mice in each group. The control group was without any treatment. The BME group received 30% (v/v) BME through drinking water. M-I group received 30 mg/kg dose of M-I once in day, every day. Body weight was monitored, and tumor size was measured using a slide caliper and volume was calculated using the formula 1/2 L X W2. After animal sacrifice tumors were dissected out and snap frozen in liquid nitrogen for further analysis. All the animal experiments were carried out in accordance NIH guidelines, following a protocol approved (1017) by the Institutional Animal Care and Use Committee (IACUC) of Saint Louis University.

### Statistical Analysis

The results are presented as means standard deviations. Data were analyzed by Student's t-test. p value of <0.05 was considered statistically significant. All experiments were repeated at least three times except animal experiments, and representative data are shown.

### EXAMPLE 1

### Identification of Active Ingredients in Bitter Melon Extract (BME)

The Inventors and others have reported the potential anticancer effect of BME in several cancer models (Dandawate et al. (2016) Chin. J. Nat. Med.; 14, 81-100; Raina et al. (2016) Semin. Cancer Biol.; 40-41, 116-129; Jia et al. (2017) Int. J. Mol. Sci.; 18, 2555; Muhammad et al. (2017) Oncotarget; 8, 66226-66236; Fang et al. (2019) Curr. Protein Pept. Sci.; 20, 296-301; Sur et al. (2020) Cancers; 12, 2064). The biological activity of BME depends on its chemical constituents. Among several ingredients in bitter melon (like phenolic acids, flavonoids, essential oils, fatty acids, amino acids, lectins, sterols, saponin and proteins), cucurbitane type triterpenoids and cucurbitane type triterpene glycosides are a major chemical class in the family Cucurbitaceae and are suggested to be responsible for bitterness and much of the biological activities in the family (Sur et al. (2020) Cancers; 12, 2064). The Inventors performed liquid chromatography coupled to high resolution electrospray ionization mass spectrometry (LC-HRESIMS) analysis to identify the cucurbitane type triterpenoids and triterpene glycosides present in the BME. The LC-HRESIMS data revealed the presence of a total of 28 secondary metabolites, of which 4 metabolites belonged to cucurbitane type triterpenoids and 20 belonged to cucurbitane triterpene glycosides in the extract (Table 1, FIG. 1A). The BME also contained a cucurbitane triterpenoid at a retention time 21.64 min with a molecular formula of C33H4807 that was not reported previously. The molecular formula and MS/MS analysis indicated this compound to be a new derivative of 7,23-dihydroxy-3-O-malonylcucurbita-5,24-dien-19-al with an extra double bond. Additionally, the HRMS analysis indicated the presence of three monoterpenoid glycosides and one oleanane-type triterpene saponin. It worth noting that many of those metabolites have unknown biological functions, otherwise reported in Table 1. Cancers 2021, 13,×3 of 14 involving c-Met and downstream signaling in both in vitro and in vivo models. This is the first study describing the therapeutic potential of M-I for the regression of HNC tumors in a pre-clinical model.

### Results

2.1. Identification of Active Ingredients in Bitter Melon Extract (BME). The Inventors and others have reported the potential anticancer effect of BME in several cancer models (Dandawate et al. (2016) Chin. J. Nat. Med.; 14, 81-100; Raina et al. (2016) Semin. Cancer Biol.; 40-41, 116-129; Jia et al. (2017) Int. J. Mol. Sci.; 18, 2555; Muhammad et al. (2017) Oncotarget; 8, 66226-66236; Fang et al. (2019) Curr. Protein Pept. Sci.; 20, 296-301; Sur et al. (2020) Cancers; 12, 2064). The biological activity of BME depends on its chemical constituents. Among several ingredients in bitter melon (like phenolic acids, flavonoids, essential oils, fatty acids, amino acids, lectins, sterols, saponin and proteins), cucurbitane type triterperinods and cucurbutance type triterpene glycodes are a major class in the family. Cucurbitaceae and are suggested to be responsible for bitterness and much of the biological activities in the family (Sur et al. (2020) Cancers; 12, 2064). The Inventors performed liquid chromatography coupled to high resolution electrospray ionization mass spectrometry (LC-HRESIMS) analysis to identify the cucurbitane type triterpenoids and triterpene glycosides present in the BME. The LCHRESIMS data revealed the presence of a total of 28 secondary metabolites, of which 4 metabolites belonged to cucurbitane type triterpenoids and 20 belonged to cucurbitane triterpene glycosides in the extract (Table 1, FIG. 1A). The BME also contained a cucurbitane triterpenoid at a retention time 21.64 min with a molecular formula of C33H4807 that was not reported previously. The molecular formula and MS/MS analysis indicated this compound to be a new derivative of 7,23-dihydroxy-3-O-malonylcucurbita-5,24-dien-19-al with an extra double bond. Additionally, the HRMS analysis indicated the presence of three monoterpenoid glycosides and one oleanane-type triterpene saponin. It worth noting that many of those metabolites have unknown biological functions, otherwise reported in Table 1.

TABLE 1
LC-HRESIMS analysis of bitter melon extract.

| Retention | Accurate | Suggested | Tentative | | |
|---|---|---|---|---|---|
| Time | Mass m/z | Formula ^{a} | Identification ^{b} | Reported Biology | Chemical Class |
| 11.72 | 949.53672 | C₄₇H₈₀O₁₉ | Momordicoside B | — | Cucurbitane triterpenoid glycosides |
| 11.95 | 969.50331 | C₄₉H₇₆O₁₉ | Goyasaponin III | — | Oleanane-type triterpene saponin |
| 12.84 | 819.47360 | C₄₁H₇₀O₁₆ | Momorcharaside A | — | Cucurbitane triterpenoid glycosides |
| 13.21 | 813.463201 | C₄₂H₆₈O₁₅ | Momordicoside O | — | Cucurbitane triterpenoid glycosides |
| 14.43 | 797.46797 | C₄₂H₆₈O₁₄ | Karaviloside X | — | Cucurbitane triterpenoid glycosides |
| 14.53 | 797.46814 | C₄₂H₆₈O₁₄ | Momordicoside N | — | Cucurbitane triterpenoid glycosides |
| 14.65 | 801.49957 | C₄₂H₇₂O₁₄ | Momordicoside C | — | Cucurbitane triterpenoid glycosides |
| 15.13 | 781.47327 | C₄₂H₆₈O₁₃ | Goyaglycoside F | — | Cucurbitane triterpenoid glycosides |
| 15.63 | 657.42089 | C₃₅H₆₀O₁₁ | Momorcharaside B | — | Cucurbitane triterpenoid glycosides |
| 16.13 | 387.20139 | C₁₉H₃₀O₈ | Vomifoliol β-D- | — | Monoterpenoid glycosides |
| | | | glucopyranoside | | |
| 16.41 | 447.22241 | C₂₁H₃₄O₁₀ | Sacranoside A | nitric oxide inhibitory | Monoterpenoid glycosides |
| | | | | effect | |
| 17.13 | 653.42599 | C₃₆H₆₀O₁₀ | Karayiloside XI | Antidiabetic | Cucurbitane triterpenoid glycosides |
| 17.54 | 635.41531 | C₃₆H₅₈O₉ | Momordicoside L | Weak α-glucosidase | Cucurbitane triterpenoid glycosides |
| | | | | inhibition | |
| 19.78 | 649.43109 | C₃₇H₆₀O₉ | Momordicoside K | — | Cucurbitane triterpenoid glycosides |
| 20.41 | 649.43114 | C₃₇H₆₀O₉ | Goyaglycoside A | — | Cucurbitane triterpenoid glycosides |
| 20.59 | 663.44660 | C₃₈H₆₂O₉ | Goyaglycoside C | — | Cucurbitane triterpenoid glycosides |
| 20.65 | 315.18017 | C₁₆H₂₆O₆ | Myrtenyl O-β-D- | — | Monoterpenoid glycosides |
| | | | glucopyranoside | | |
| 20.74 | 615.38922 | C₃₆H₅₄O₈ | Charantoside VII | — | Cucurbitane triterpenoid glycosides |
| 20.80 | 619.42041 | C₃₆H₅₈O₈ | Momordicoside V | | Cucurbitane triterpenoid glycosides |
| 20.95 | 631.42040 | C₃₇H₅₈O₈ | Charantoside I | — | Cucurbitane triterpenoid glycosides |
| 21.05 | 633.43618 | C₃₇H₆₀O₈ | Charantoside V | — | Cucurbitane triterpenoid glycosides |
| 21.06 | 473.3627 | C₂₀H₄₈O₄ | Momordicine I | stimulate insulin | Cucurbitane-type triterpene |
| | | | | secretion in vitro | |
| | | | | diabetes- | |
| | | | | associated | |
| | | | | cardiac fibrosis. | |
| 21.28 | 635.4518 | C₃₇H₆₂O₈ | Karaviloside III | cytotoxic activity | Cucurbitane triterpenoid glycosides |
| | | | | against Hep3B and | |
| | | | | HepG2 cell lines | |
| 21.54 | 649.46745 | C₃₈H₆₄O₈ | Karaviloside II | — | Cucurbitane triterpenoid glycosides |
| 21.64 | 557.34720 | C₃₃H₄₈O₇ | No hit-new | — | Cucurbitane-type triterpene |
| | | | malonylcucurbita- | — | |
| | | | trien-19-al | | |
| | | | derivative | | |
| 21.72 | 559.36299 | C₃₃H₅₀O₇ | 7,23-Dihydroxy-3-O- | — | Cucurbitane-type triterpene |
| | | | malonylcucurbita- | | |
| | | | 5,24-dien-19-al | | |
| 21.91 | 601.40955 | C₃₆H₅₆O₇ | Charantoside IV | | Cucurbitane triterpenoid glycosides |
| 22.60 | 437.34125 | C₃₀H₄₄O₂ | (23E)-Cucurbita- | — | Cucurbitane-type triterpene |
| | | | 5,23,25-triene-3,7- | | |
| | | | dione | | |

### EXAMPLE 2

### M-I Inhibits HNC Growth In Vitro

Due to their limited reported biology, the Inventors have selected a few triterpenoids and triterpene glycosides, based on their availability, for preliminary in vitro screening against HNC (JHU022, JHU029, Ca127) cell lines. The Inventors identified momordicine-I (M-I) as one of the chemical constituents at the retention time of 21.06 min in the BME (FIG. 1A). The M-I [C30H4804, PubChem CID: 14807332; IUPAC Name: (3S, 7S, 8S, 9R, 10R, 13R, 14S, 17R) 3,7-dihydroxy-17-(4-hydroxy-6-methylhept-5-en-2-yl)-4,4,13,14-tetramethyl-2,3,7,8,10,11, 12,15, 16,17-decahydro-1H-cyclopenta[a]phenanthrene-9-carbaldehyde] is a white crystalline solid that belongs to the cucurbitane-type triterpene class (FIG. 1B). The Inventors performed cytotoxicity assays of M-I in HNC cells Ca127, JHU029 and JHU022 and observed that M-I inhibited HNS cells in a dose-dependent manner (FIG. 1C). However, M-I had a minimum effect on human normal oral keratinocytes (NOK). The IC50 doses of Ca127, JHU022 and JHU029 cells treated with M-I for 48 h were 7 ug/mL, 17 ug/m L and 6.5 ug/mL, respectively. Other compounds, mom ordicoside K (MK), and karavilagenin D (KD), were also examined on HNC cell cytotoxicity assay. These, two other compounds required much higher amounts (>50 ug/mL) even for -40% cell death.

### EXAMPLE 3

M-I Inhibits c-Met Signaling in HNC Cells

The Inventors observed that BME inhibited c-Met signaling and its downstream signaling molecules to prevent HNC growth (Rajamoorthi et al. (2013) PLoS ONE; 8, e78006). To investigate the mechanistic effect of M-1, Ca127, JHU029 and JHU022 cells were treated with M-I for 48 hr along with vehicle control. BME was used in parallel as a control. A significant reduction of c-Met expression was observed in these cells following BME or M-I treatment (FIG. 2A). The receptor tyrosine kinase c-Met induces tumor development by activating multiple downstream molecules, including the oncogenic transcription factor signal transducer and activator of transcription 3 (STAT3) (Kermorgant et al. (2008) J. Cell Biol.; 182, 855-863; Organ et al. (2011)Ther. Adv. Med Oncol., 3, S7—S19. Upon activation of c-Met signaling, STAT3 is activated by phosphorylation at the Tyr-705 residue, dimerized and translocated to the nucleus for activation of several proliferation and survival related genes including c-Myc, survivin and cyclin D1 Organ et al. (2011)Ther. Adv. Med Oncol., 3, S7—S19; Akiyama et al. (2011) Int. J. Oncol.; 38, 1245-1252). Activated STAT3 can also transcribe STAT3 gene as a positive feedback mechanism (Akiyama et al. (2011) Int. J. Oncol.; 2011, 38, 1245-1252). The Inventors observed a significant reduction in phospho-STAT3 (Tyr-705) expression following treatment with BME or M-I in Ca127 and JHU029 cells (FIG. 2B). A substantial reduction in total STAT3 level was also noted in M-I treated cells. Next, the Inventors examined whether the short term (15, 30, 60, 120, 240 min) BME or M-I treatment can inhibit p-STAT3 levels to ascertain whether initially M-1 inhibits p-STAT3 levels without affecting total STAT protein levels. Interestingly, the Inventors have observed that BME or M-I treatment on HNC cells inhibits pSTAT3 in 15 min without affecting total STAT3, however, after 240 min of treatment, total STAT3 was inhibited (FIG. 2C). This may be due to the feedback mechanism of pSTAT3 on STAT3 regulation as discussed earlier. Id. Further, depletion of c-Met by specific siRNA inhibits pSTAT3 (FIG. 2D). The Inventors also observed a significant inhibition of STAT3 downstream molecules; c-Myc survivin and cyclin D1, in Ca127 and JHU029 cells following treatment with BME and M-I (FIG. 3). Taken together, the Inventors' results suggested that M1 inhibits c-Met signaling in the prevention of HNC growth (FIG. 4).

### EXAMPLE 4

### Pharmacokinetic and Toxicity Profile of M-I

To evaluate the pharmacokinetic (PK) profile of M-I, a single dose (20 mg/kg) was given to C57131/6 male mice by either intraperitoneal injection (IP group; n =3) or oral ga-vage (oral group; n=3). A dose of 20 mg/kg was chosen based on previous publications (Asensi et al. (2002) Free. Radic. Biol. Med.; 33, 387-398; Qiao et al., (2014) Pharm. Biol.; 52, 228-236). The average plasma concentration standard deviation at each time point is shown in FIG. 5A and corresponding PK parameters are summarized in Table 2. M-I was rapidly absorbed with a maximum plasma concentration 1 h post-IP and PO dose. Cmax values were 18 M and 0.5 M after the single 20 mg/kg IP and PO dose, respectively. The observed elimination half-life was 0.9 h in the IP group and 2 h in the PO group. The oral group had loose stool, starting four hours post-dose, which had not re-solved by 8 h but all mice appeared normal after twenty-four hours. No adverse events were observed for the mice in the IP dosing group. The Inventors therefore selected IP administration of M-I for subsequence studies.

For toxicity analysis, mice (n=3) were given 20 mg/ kg of M-I twice a day or 30 mg/kg IP dose of M-I once a day by IP injection for five days. In parallel, there were untreated control (n=3) and BME treated group (n=3) for comparison. Behavior and body weight of mice were monitored daily. The body weight in all the mice was stable, with no drastic changes seen in any of the mice tested. The Inventors collected blood on day six, and serum chemistries related to liver and kidney function were examined. The concentration of total bilirubin, alanine transaminase, aspartate transaminase, alkaline phosphatase, creatinine, urea, and glucose were comparable in M-I and BME treated groups with untreated control mice, indicating no toxic effect (FIG. 5B).

TABLE 2
Pharmacokinetics parameters of momordicine I (M-I) administered either
through intraperitoneally (IP) or orally (PO) in C57BI/6 male mice
(data provided as mean ± SD).

| Pharmacokinetic | C57BI/6 Male | |
|---|---|---|
| Parameters | 20 mg/Kg-IP | 20 mg/Kg-PO |
| T_{1/2} (h) | 0.90 ± 0.02 | 2.11 ± 0.3 |
| Tₘₐₓ (h) | 1.00 ± 0 | 1.00 ± 0.9 |
| Cₘₐₓ (ng/mL) | 8427 ± 3419.4 | 214 ± 18.6 |
| Cₘₐₓ (μM) | 17.83 ± 7.2 | 0.45 ± 0.04 |
| AUCₗₐₛₜ (min*ng/mL) | 762,559 ± 319,312.5 | 38,584 ± 16,628.4 |
| AUC_{INF}_obs (min*ng/mL) | 765,026 ± 320,184.6 | 41,251 ± 17,124.75 |
| AUC (% Extrap) | 0.33 ± 0.08 | 6.84 ± 1.5 |

Abbreviations: T_{1/2}: Elimination half-life; Tmax: time to reach maximum (peak) plasma concentration following drug administration; Cmax: maximum (peak) plasma drug concentration; AUClast: area under the plasma concentration-time curve from time zero to time of last measurable concentration; AUCINF_obs: area under the concentration-time curve extrapolated from zero up to infinity; AUC (% Extrap): area under the first moment of the plasma concentration-time curve extrapolated from time t to infinity as a percentage of total AUC; Cl_obs: apparent total body clearance of the drug from plasma.

### EXAMPLE 5

### Therapeutic Potential of M-I in HNC Xenograft Model

The Inventors investigated the therapeutic efficacy of M-I in HNC xenograft model. For this, The Inventors implanted JHU029 cells in flanks of nude mice. After the formation of a palpable tumor, mice were divided into three groups: untreated control (n=5), BME treated group (n=5), and M-I treated group (n=5). In the BME group, mice were given 30% BME through drinking water as described previously (Sur et al. (2017) Cancer Prey. Res.; 11, 191-202). The mice in M-I group received 30 mg/kg of M-I through IP, once a day till the end of the experiment. Body weight and tumor volume were measured. The Inventors observed a sudden drop in body weight (−10%) in three out of five mice in M-I group on Day 20; however, they gained back the weight (FIG. 6A). The Inventors also observed the formation of fluid in the tumors as reported previously (Rajamoorthi et al. (2013) PLoS ONE; 8, e78006; Sur et al. (2019) Cell Commun. Signal.; 17, 131). Some of the tumors in the control group had an open wound; therefore, the Inventors needed to sacrifice all the animals on day 32. The BME and M-I group mice displayed significantly reduced tumor volume (FIG. 6B, C). However, M-I showed a better effect in reducing tumor growth. The Inventors examined c-Met signaling in control and treated tumors. The Inventors observed a significant reduction in expression of c-Met and its downstream molecule c-Myc in BME and M-I treated groups compared to untreated control tumors (FIG. 6D).

For further validation, the Inventors also examined the effect of M-I in Ca127 xenograft model. The Inventors observed increasing body weight in all the mice throughout the experiment, and −50% reduction of tumor growth in BME or M-I treated xenograft tumor (FIG. 7A, B). The Inventors further observed a significant reduction in c-Met and c-Myc expression in BME and M-I treated groups compared to the control (FIG. 7C, D). the Inventors' results demonstrated that M-I has the potential as a therapeutic candidate for HNC treatment.

### SUMMARY

In summary, this was the first report demonstrating M-I, a secondary metabolite from bitter melon, inhibited tumor growth in HNC xenograft models with no apparent toxicity. Mechanistic data demonstrated that M-I impairs c-Met signaling in HNC (JHU022, JHU029, Ca127) cells, which is schematically illustrated in FIG. 4. Thus, M-I may be used as a chemotherapeutic agent alone or in combination with other chemotherapeutic agents against HNC.

### EXAMPLE 6

M-I inhibits TNBC Cell Growth in vitro.

The Inventors tested human MDA-MB-231 and mouse 4T1 cells in dose dependent manner (FIG. 8). Treatment of MDA-MB-231 and 4T1 cells with M-I showed the IC50 value is 12 ug/ml and 5 ug/ml, respectively. The Inventors also tested other compounds in TNBC cell cytotoxicity assays, but these required much higher amounts (>50 pg/m L) even for ˜40% cell death. The Inventors further performed cytotoxicity assays of M-I using the TNBC cell lines MDA-MB-231, BT459 and MDA-MB-468 and observed that M-I inhibits cell growth in a time-dependent manner. However, M-I had a minimum effect on human normal mammary epithelial cells.

### EXAMPLE 7

### M-I Enhances NK-Cell-Mediated Cytotoxicity in MDA-MB-231 Cells.

NK cells are implicated in drug induced resistant cancer cells to drive tumor growth (Smalley et al., 2020). To interrogate the activity of NK cells in TNBC cells following M-I treatment, the Inventors initially exposed NK (NK3.3) cells to bitter melon extract or M-I for 48h or 72h and observed no cytotoxic effect on NK-cell viability (FIG. 9). Bitter melon extract was used as a control. Control or M-I treated NK3.3 cells were also cocultured with MDA-MB-231 cells at 1:5 (Target:Effector) ratio for 24 h and stained for viable/dead cells. M-I-treated NK cells showed enhanced cytotoxicity as compared to IL-2 activated or bitter melon extract treated NK cells (FIGS. 10A and 10B). The apoptotic markers were further examined by Western blot analysis. The Inventors found that M-I treated NK cells, when co-cultured with MDA-MB-231 cells, activate caspase 9/3 and PARP cleavage in breast cancer cells (FIG. 11). Similar results were obtained from BT549 cells treated with M-I/NK cells.

### EXAMPLE 8

M-I inhibits Breast Cancer Growth in tRiple Negative Breast Cancer Mouse Models.

The Inventors further evaluated the efficacy of M-I in orthotopic breast cancer mouse models in a pilot study. Syngeneic breast cancer model using 4T1 (mouse) cells implanted into the mammary fat pad of Balb/c mice. Mice were divided into two groups. One group received M-I (30 mg/mouse/day) and the other group received vehicle as a control by ip. Tumor volumes were significantly reduced in M-I treated groups compared to the control group (FIG. 12).

In summary, the Inventors have demonstrated that M-I acts as a therapeutic agent for controlling breast tumor growth in preclinical model. It also exerts its effect in multiple ways which also include activating NK cell function for killing breast tumor cells.

### CONCLUSIONS

The Inventors have demonstrated M-I, a secondary metabolite from bitter melon, inhibited tumor growth in HNC xenograft models as well as a breast cancer model. M-1 inhibited c-Met signaling and was demonstrated to activate NK function for killing breast tumor cells. Furthermore M-1 exerts it's affects with no apparent toxicity. Thus, M-I may be used as a chemotherapeutic agent alone or in combination with other chemotherapeutic agents against HNC, breast cancer or any other solid tumors to which administration is achieved.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed:

1. A method of treating a subject with a solid tumor, the method comprising:
a) selecting a subject diagnosed with a solid tumor;
b) administering to the subject a pharmaceutical acceptable composition comprising one or more lipophilic solvents selected from the group consisting of: DMSO, ethyl acetate, acetone, methanol, and dichloromethane; and an effective amount of Momordicine-I.

2. The method of claim 1 whereas, the solid tumor is selected from the group consisting of: head and neck cancer and breast cancer.

3. The method of claim 1 whereas one or more symptoms of the solid tumor are diminished.

4. The method of claim 1 whereas one or more indicators of solid tumor are diminished.

5. The method of claim 4 whereas one or more indicators are selected from the group consisting of C-Met, pSTAT3, and ki67.

6. The method of claim 1 whereas the lipophilic solvent is DMSO.

7. The method of claim 1 whereas the composition further comprises a cyclodextrin.

8. The method of claim 7 wherein the cyclodextrin consists of Captisol.

9. The method of claim 1 whereas administration is by interperitoneally injection.

10. The method of claim 1 whereas administration is by intravenously injection.

11. The method of claim 1 whereas administration is by intramuscularly injection.

12. The method of claim 1 whereas administration is oral.

13. The method of claim 1 whereas administration is repeated once or more daily.

14. The method of claim 1 whereas an effective amount is about 30 mg/kg.

15. The method of claim 1 whereas an effective amount is about 20 mg/kg.

16. The method of claim 1, whereby the subject is administered a daily 30 mg/kg dose of M-I treatment by intravenous injection using a treatment solution of 2 mg M-I /mL dissolved in 5% DMSO/95% of a 30% w:v Captisol solution.

17. A composition for the treatment of solid tumors, the composition comprising:
a) a pharmaceutical acceptable composition comprising one or more lipophilic solvents selected from the group consisting of: DMSO, ethyl acetate, acetone, methanol and dichloromethane; and
b) an effective amount of Momordicine-1.

18. The composition of claim 17, further comprising a cyclodextrin.

19. The composition of claim 18, wherein the cyclodextrin consists of Captisol.

20. A composition for the treatment of head and neck cancer, the composition comprising 2 mg Momordicine-I/mL dissolved in 5% DMSO/95% of a 30% w:v Captisol solution.
